(19) United States
(12) Patent Application Publication
(10) Pub. No.: US 2013/0261580 A1
(43) Pub. Date: Oct. 3, 2013

(54) SINGLE-LAYER ANTIMICROBIAL DISPOSABLE SWIM DIAPER

(71) Applicant: Patti C. Gilmer, Lula, GA (US)

(72) Inventor: Patti C. Gilmer, Lula, GA (US)

(21) Appl. No.: 13/437,747

(22) Filed: Apr. 2, 2012

(51) Int. Cl. A61F 13/494; A61L 15/18

(52) U.S. Cl. 604/367; 604/385.29

(57) ABSTRACT

A swim diaper for use by a child while swimming, bathing or otherwise exposed to an aqueous environment includes a main torso section formed from a sheet of breathable, waterproof material which has been treated with an antimicrobial solution so as to prevent the spread of recreational water illnesses. The material is of sufficient thickness to retain solid waste and having a moisture vapor transmission rate of at least 200 g/m²/day. The main torso section has a torso receiving aperture at the upper end of the main torso section for receiving a torso and leg receiving apertures located in the lower portion of the main torso section for receiving a child's legs.

### BACKGROUND OF THE INVENTION

1. Field of the Invention

This invention relates generally to a garment for preventing the spread of germs, bacteria, and viruses. More specifically, this invention relates to a single-layer, non-absorbent swim diaper treated with an antimicrobial solution to prevent the spread of recreational water illnesses.

2. Description of the Related Art

In recent decades, highly absorbent disposable diapers have replaced reusable cotton diapers as the diaper of choice for use by most parents. This is in large part due to the fact that disposable diapers are generally much more absorbent than cloth diapers, are easier to use, and may be conveniently discarded when soiled. However, neither reusable cloth diapers nor conventional disposable diapers are well adapted for use by infants or small children while swimming or participating in other water activities.

When used during water activities, highly absorbent disposable diapers and thick reusable cloth diapers become bulky and can pose a potential drowning hazard to infants and small children. Since these standard diapers are designed to absorb large quantities of water, they may become quite heavy while a child is swimming, hindering the child's ability to stay afloat. These diapers may also become a falling hazard for toddlers once they have left the water due to their increased weight and bulkiness.

In addition, conventional absorbent disposable diapers become uncomfortable when saturated with water, and may fall off of a child due to the increased saturated diaper weight. This creates a need for parents to frequently change the diapers of children who are swimming or bathing, resulting in significant inconvenience, use of a large number of diapers, and thus increased cost.

Also, many disposable diapers are constructed of fibrous materials which may quickly disintegrate in water. Failure of these diapers can result in the introduction of loose fibrous material into the pool water, creating a possible health hazard if swallowed, for example, as well as potentially clogging pool filtration and cleaning equipment, leading to increased pool cleaning and maintenance costs.

Failure of these diapers can also result in the introduction of fecal waste into the pool water, not only creating a possible health hazard for users of the pool, but also creating an unsightly and unpleasant swimming environment. Fecal solid waste and diarrhea in swimming pool water may lead to one of several recreational water illnesses (RWIs) caused by swallowing contaminated pool water. RWIs are caused by germs such as Cryptosporidium, Giardia, E. coli, and Shigella. Although chlorine kills several of the germs associated with RWIs, it does not kill the germs immediately. As a result, pools generally need to be closed until necessary disinfection measures can be taken in response to the fecal incident. Pool closures invariably present a major inconvenience to pool operators and swimmers alike. Thus, there is a need for ways to reduce the likelihood that children using swimming pools will contaminate the pool with fecal matter.

One approach to providing a diaper for use by swimming infants is to combine a water-tight seal around the diaper openings in conjunction with an outer waterproof layer to prevent the absorbent portion of the diaper from coming into contact with water in the surrounding environment. Many conventional diapers have used elastic in an attempt to create a water-tight seal between the diaper and the wearer along the leg and body openings of the diaper.

U.S. Pat. Nos. 4,585,447 and 4,643,728 disclose disposable diapers having an absorbent layer encased within two hydrophobic outer sheets and the use of elastic crotch and waist seals to prevent leakage of liquid waste from the diapers. However, it is difficult to use elastic to maintain a seal sufficiently tight between a diaper and the skin of a small child to prevent the absorbent material from coming into contact with the water without unacceptably constricting the torso and extremities of the child. Excess constriction may interfere with the child's comfort and mobility, and potentially may cause circulation problems in the extremities of the child.

Another approach to the problem is to encase the diaper in a waterproof material and create a water-tight seal to protect a diaper from the water while a child is swimming. For example, U.S. Pat. No. 5,502,842 discloses a water-proof protective swimwear garment designed to be worn over a conventional diaper. The invention uses inflatable chambers at the torso and leg openings of the garment to produce a water-tight seal and prevent the underlying diaper from coming into contact with the water. Unfortunately, this approach requires the presence of buoyant air-filled chambers at the child's abdomen and legs, shifting the child's center of buoyancy lower within the body and thereby making it difficult for a child to maintain his or her head above the surface of the water. Additionally, use of a waterproof material prevents escape of sweat and may lead to development of a rash or chafing of the areas of skin enclosed by the garment.

U.S. Pat. No. 6,195,800, the specification of which is incorporated herein by reference, discloses a swim diaper for use by a child while swimming, bathing or otherwise exposed to an aqueous environment while U.S. Pat. No. 6,797,856 discloses an absorbent swimwear garment having a biocidal agent attached to the garment for the purpose of killing or immobilizing microorganisms. However, like other conventional absorbent disposable diapers, the garment disclosed in the '856 patent may become uncomfortable when saturated with water and may fall off of a child due to the increased saturated diaper weight. In addition, the garment disclosed in this patent is a multi-layered product which further adds to weight as well as its complexity and cost.

In light of the above disadvantages of prior diaper products, it would be advantageous to provide a diaper designed specifically for use during water activities which does not pose a safety risk when saturated, is comfortable to wear when wet, does not need to be frequently changed when exposed to water, and reduces the likelihood of pool contamination due to a fecal incident.

It is therefore an object of the present invention to provide a new and improved diaper to retain potentially hazardous liquid and solid waste, which will not become bulky or heavy through absorption of large quantities of liquid when a child is swimming, bathing or otherwise immersed in an aqueous environment.

It is another object of the present invention to provide a new and improved diaper product which may be comfortably worn under the outer swimwear of a swimming or bathing child and will remain comfortable even following immersion in water.

It is another object of the present invention to provide a new and improved diaper product which is waterproof and breathable and will thus remain comfortable even following immersion in water.

It is yet another object of the present invention to provide a new and improved diaper which will neither disintegrate nor fall off of a child following immersion in water and thus does not need to be frequently changed while a child is bathing or otherwise exposed to an aqueous environment.

It is still another object of the present invention to provide a new and improved diaper which prevents or reduces the spread of bacteria and viruses when used in an aqueous environment.

### BRIEF SUMMARY OF THE INVENTION

The above objectives are accomplished according to the present invention by providing a swim diaper for use by a child while swimming, bathing or otherwise exposed to an aqueous environment. The swim diaper includes a main torso section formed from a single layer of breathable, waterproof, non-absorbent, non-woven material which has been treated with an antimicrobial solution, is of sufficient thickness to retain solid waste, and has a moisture vapor transmission rate of at least 200 g/m²/day. This main torso section has a torso receiving aperture at the upper end of said main torso section for receiving a torso and leg receiving apertures located in the lower portion of said main torso section for receiving a child's legs.

### BRIEF DESCRIPTION OF THE DRAWINGS

The construction designed to carry out the invention will hereinafter be described together with other features thereof. The invention will be more readily understood from a reading of the following specification and by reference to the accompanying drawings forming a part thereof, wherein an example of the invention is shown.

FIG. 1 is diagram illustrating a child wearing a swim diaper, according to the present invention, under a swimsuit.

FIG. 2 is an exploded perspective view illustrating the structure of the swim diaper according to the present invention.

FIG. 3 is a cross-section taken along lines 3--3 in FIG. 1 illustrating the structure of the swim diaper according to the present invention.

### DETAILED DESCRIPTION OF THE INVENTION

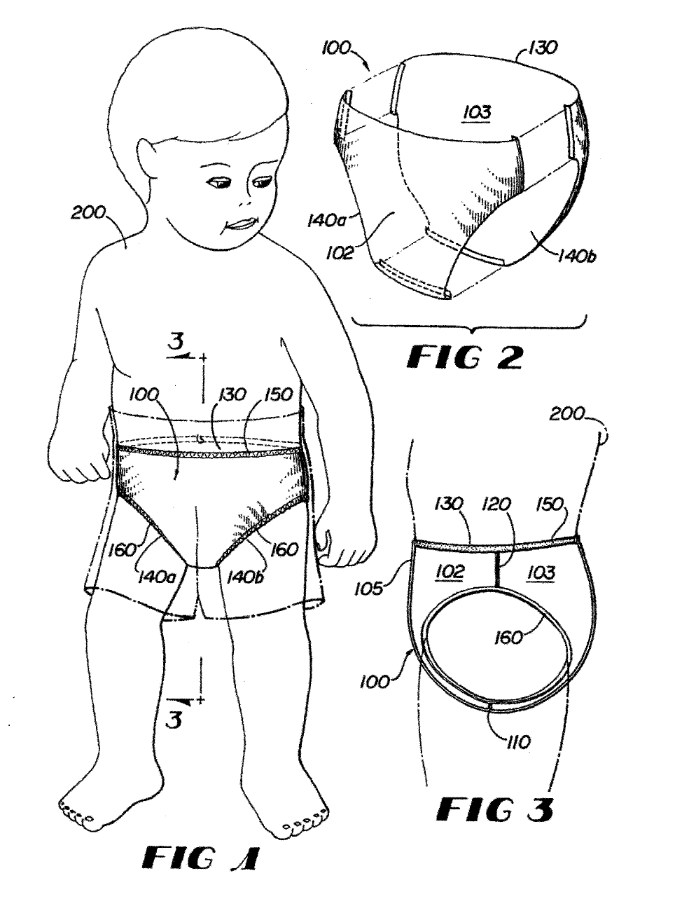

Referring now in more detail to the drawings, the invention will now be described in more detail. Referring to FIG. 1, the swim diaper 100 may be worn by a child 200 while swimming or otherwise immersed in an aqueous environment, such as while bathing or playing with a hose or lawn sprinkler.

Referring now to FIG. 2 and FIG. 3, swim diaper 100 may be made from a relatively inexpensive fabric and is thus disposable, although a more sturdy fabric may be used to construct a reusable swim diaper. The fabric is thin enough to be worn comfortably under a child's outer swimwear while at the same time having sufficient structural integrity to contain solid waste. The swim diaper 100 is waterproof and substantially non-absorbent to minimize absorption of liquid waste or liquids from the surrounding environment. Because the fabric is waterproof, no appreciable weight is added by the absorption of bodily fluids or water from the surrounding environment, as would occur with cotton diapers or standard disposable diapers. Furthermore, the swim diaper 100 is breathable to enhance the comfort of the wearer.

In a preferred embodiment, the swim diaper 100 is constructed from a medical grade, waterproof, breathable laminate such as Bertek Medifilm 330®. Bertek Medifilm 330® is a waterproof laminate consisting of a thin layer of DuPont Hytrel® polyester film coating disposed on a supporting layer of DuPont Sontara® which is a non-woven, spun laced polyester fabric. Bertek Medifilm 330® is breathable, having a moisture vapor transmission rate of approximately 500 to 570 g/m²/day. Alternatively, a preferred embodiment of the present invention relates to a disposable, non-absorbent swim diaper manufactured from a single layer of SMMS (spunbond-meltblown-meltblown-spunbond) nonwoven material such as polypropylene commonly used in the production of disposable medical wear and linen.

For the comfort of the child it is important that the laminate be waterproof, substantially non-absorbent, and breathable, with a moisture vapor transmission rate of at least 200 g/m²/day. The necessary moisture vapor transmission rate will, however, vary depending upon the nature of the non-woven supporting material and preferably should be below approximately 2400 g/m²/day. Of course, one of ordinary skill in the art will recognize that the swim diaper of the present invention may also be constructed of other suitable breathable, waterproof materials with sufficient strength. Examples of alternative materials include treated polyester, acrylic, nylon, or polypropylene fabrics and laminates.

In accordance with the present invention, the nonwoven material used to form the swim diaper 100 is treated with a silver-based antimicrobial agent. One example of a commercially available silver-based antimicrobial solution is SilverDur™ available Dow Chemical. The antimicrobial agent is preferably a solution which may be applied by any conventional technique known to those skilled in the art such as by spraying, foaming, dipping, soaking, saturation, or otherwise coating the swim diaper 100 with a silver-based antimicrobial agent. Thus, the swim diaper of the present invention is designed to prevent or reduce exposure to many of the bacteria and viruses encountered in swimming pools, hot tubs and the like. Optionally, once treated with the antimicrobial solution, the nonwoven fabric may be provided with a lamination layer.

In the preferred embodiment, swim diaper 100 consists of a sheet of laminate with a thickness of approximately 1-3 mm cut to form a standard symmetrical pattern for underwear having a front main section 102 and a rear main section 103. When sewn at the crotch 110 and hips 120, these two sections form an main torso section 105 of an undergarment having a waist receiving opening 130 and two leg receiving openings 140a and 140b. An elastic waist band 150 and elastic leg bands 160 may be sewn into these openings to ensure a snug yet comfortable fit.

The front and rear main sections of the swim diaper 102 and 103 may be joined at the crotch 110 and hip 120 seams by being either sewn, secured with adhesive or by heating the foam under pressure to cause the plastic to bond by thermoplastic lamination. Of course, it will be clear to one of ordinary skill in the art that any of a number of standard undergarment sewing patterns may be used to construct the swim diaper 100 and that the seams may be joined by any equivalent method.

The swim diaper 100 may be used by first garbing the child and ensuring that there is a snug fit of the elastic waist band and elastic leg bands. The child may then be allowed to swim, bathe, play in a sprinkler or perform any other water activities. Any solid or semi-solid waste emissions will be contained by the diaper. In addition, because an antimicrobial agent has been applied to the swim diaper 100, the potential for bacteria and viruses to escape from the swim diaper 100 alive or active and continuing to Live will be prevented or significantly reduced. When the child has completed his exposure to the aqueous environment the diaper may be removed and either discarded or washed and reused.

In order to demonstrate the effectiveness of the single-layer, antimicrobial, disposable swim diaper 100 of the present invention, a number of tests were conducted. The results of one such test, the dynamic shake flask test, are provided in the table below. This test method was used to demonstrate the antimicrobial effect of a sample of material treated with a silver-based antimicrobial agent as taught by the present invention as compared to an inoculum only control sample.

| | The number of bacteria recovered | | | |
|---|---|---|---|---|
| | from (CFU/mL) | | | |
| | | | “24 h” | |
| Name of | | “0” contact | contact | Reduction |
| test organism | | time | time | % |
| Escherichia coli | Sample (1 g) | | <30 | >99 |
| ATCC 25922 | Inoculum | 2.9 × 10⁵ | 5.5 × 10⁶ | |
| | only control | | | |
| Shigella flexneri | Sample (1 g) | | 52 | >99 |
| CMCC(B)51571 | Inoculum | 1.6 × 10⁵ | 2.2 × 10⁶ | |
| | only control | | | |
| Salmonella | Sample (1 g) | | <30 | >99 |
| typhimurium | Inoculum | 2.8 × 10⁵ | 4.3 × 10⁶ | |
| AS1.1194 | only control | | | |

The antimicrobial effect of a silver-based antimicrobial agent in accordance with the teachings of the present invention was measured after a dwell time of 24 hours. Each sample was evaluated for antimicrobial efficacy against Escherichia coli, Shigella flexneri, and Salmonella typhimurium. Antimicrobial performance of each sample was evaluated in accordance with the dynamic shake flask test (ASTM E2149). Specifically, each sample was placed in individual receptacles containing the inoculum. Then, each sample was placed on a shaker for 24 hours. An organism count was then made on each sample as well as the inoculum and the percent reduction (based on the original inoculum) determined. The results of the test for each sample are presented in the table above. As can be seen, the material used to form the disposable swim diaper 100 of the present invention displayed excellent antimicrobial performance with kill rates greater than 99%.

What have been described above are preferred embodiments of the present invention. It is, of course, not possible to describe every conceivable combination of methodologies for purposes of describing the present invention. However, one of ordinary skill in the art will recognize that many further combinations, permutations, modifications and applications of the present invention are possible. For example, the swim diaper of the present invention may be used both in and out of the water. Also the swim diaper of the present invention may be used alone, as an undergarment under another garment, or as a cover over another garment such as panties or underwear, for example, to protect sheets, bedding, chairs, car seats, flooring and the like. In addition, the swim diaper of the present invention is not limited to infants and small children. Those of ordinary skill in the art will appreciate that the present invention may be used with adults such as the elderly who may experience incontinence. Therefore, all such possible combinations, permutations and modifications are to be included within the scope of the claimed invention, as defined by the claims below.

What is claimed is:

1. A single-layer swim diaper for use by a child when swimming, bathing or otherwise exposed to an aqueous environment, said diaper consisting essentially of:
a main torso section formed from a laminate, said laminate comprising a film of breathable, waterproof material bonded onto a substrate of non-woven, substantially non-absorbent fabric having a thickness sufficient to retain solid waste, wherein said laminate has a thickness in the range of one millimeter to three millimeters, and a moisture vapor transmission rate of at least 200 g/m²/day;
said main torso section having a torso receiving aperture at the upper end of said main torso section for receiving a torso, said torso receiving aperture including an elastic band to ensure a snug fit about said torso;
said main torso section having leg receiving apertures located in the lower portion of said main torso section for receiving legs, each of said leg receiving aperture including an elastic band to ensure a snug fit about a respective leg; and
an antimicrobial solution disposed on the swim diaper.

2. The swim diaper of claim 1, wherein said laminate has a moisture vapor transmission rate of less than 2400 g/m²/day.

3. The swim diaper of claim 1, wherein said laminate has a moisture vapor transmission rate in the range of 500 to 570 g/m²/day.

4. The swim diaper of claim 1, further comprising elastic bands carried by the periphery of said torso and leg receiving apertures for ensuring a snug fit, thereby restricting the passage of bacteria into the aqueous environment.

5. The swim diaper of claim 1, wherein said substrate includes a spun laced polyester.

6. The swim diaper of claim 5, wherein said substrate is generally 1-3 mm thick.

7. The swim diaper of claim 1, wherein said main torso section further comprises two sheets of laminate joined at the crotch and hips to form a standard underwear pattern.

8. The swim diaper of claim 7, wherein said sheets of laminate are joined by stitches.

9. A breathable, waterproof, single layer swim diaper for use to retain solid or semi-solid waste excreted by swimming children, said swim diaper consisting essentially of:
a main torso section formed from a laminate, said laminate comprising a film of breathable, waterproof material bonded onto a substrate of non-woven, substantially non-absorbent fabric having a thickness sufficient to retain solid waste;
said main torso section having a torso receiving aperture located at the upper end of said main torso section;
said main torso section having leg receiving apertures located in the lower portion of said main torso section;
elastic bands carried by the periphery of said torso and leg receiving apertures to ensure that said apertures snugly fit the legs and torso of the wearer; and
an antimicrobial solution disposed on the swim diaper.

10. A single-layer garment, comprising:
a main torso section formed from non-woven, substantially non-absorbent material treated with a silver-based antimicrobial agent;
said main torso section having a torso receiving aperture at the upper end of said main torso section for receiving a torso, said torso receiving aperture including an elastic band to ensure a snug fit about said torso; and
said main torso section having leg receiving apertures located in the lower portion of said main torso section for receiving legs, each of said leg receiving apertures including an elastic band to ensure a snug fit about a respective leg.
